(19) United States
(12) Patent Application Publication
(10) Pub. No.: US 2011/0017213 A1
(43) Pub. Date: Jan. 27, 2011

(54) Surgical and Anesthesia Conduit Cover Kit and Method

(76) Inventor: Mark V. Vadney, Altus, OK (US)

Correspondence Address: JAMES W. PRAVEL;PRAVEL INTELLECTUAL PROPERTY LAW, PC, 1800 DIAGONAL ROAD, SUITE 600, ALEXANDRIA, VA, 22314, US

(21) Appl. No.: 12/508,627

(22) Filed: Jul. 24, 2009

(51) Int. Cl. A61M 16/01; B23P 11/00

(52) U.S. Cl. 128/204.17; 29/428

(57) ABSTRACT

A disposable anesthesia conduit cover kit that includes a dispenser and pre-measured, or cut to length tubing segments with fasteners to secure each end of the tubing segment. The dispenser may be wall mounted or lay flat and alternative dispenser configurations are provided. The tubing segments protect the anesthesia conduit cover from blood or body fluids to protect patients and healthcare workers from infectious and pathogenic microorganisms and to assist with the compliance of both state and federal regulations.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO MICROFICHE APPENDIX

Not applicable.

### FIELD OF THE INVENTION

This invention relates to the prevention of the growth and/or spread of bloodborne pathogens and in particular involves a kit for preventing the spread of bloodborne pathogens from forced air warming unit accordion-type hoses.

### BACKGROUND OF THE INVENTION

Much research has been conducted into the subject of blood-borne contamination and the transmission of infectious diseases in the surgical environment. Many guidelines have been formulated by multiple agencies and organizations. The available scientific evidence reflects that these dictums are ineffective and impractical. Pathogenic microorganisms have been cultured from various sources in the operating room before and after disinfection. Anesthesia equipment has been cited as reservoirs for bacterial colonization. Some investigators have proposed the necessity of anesthesia equipment design changes that will increase the ease and effectiveness of adequate disinfection. Single use anesthesia devices are one potential solution.

The rate of new infections that patients acquire while hospitalized in the United States is staggering; it is suggested that 10% of hospital inpatients will be victims of nosocomial infection (as cited in Loftus et al., 2008), with the rate in Canada perhaps slightly higher. There is strong evidence suggesting that the incidence and associated risk are only increasing, with an annual mortality conservatively estimated at many tens of thousands of patients. See AANA Journal, June 2009, Vol. 77, No. 3, page 230.

The prevention of the transmission of blood-borne pathogens (Human Immunodeficiency Virus and Hepatitis B) in the workplace was the impetus for the United States Department of Labor's Occupational Safety & Health Administration (“OSHA”) Regulations for Bloodborne pathogens (1991). As the risk for blood and body fluid exposure for healthcare workers is extraordinary, safety and exposure prevention in the healthcare setting is imperative. These concerns were the foundation for recommendations on hospital infection control and decontamination procedures from various regulatory agencies. However, concerns over the increasing prevalence of antibiotic resistant microorganisms such as methicillin resistant staphylococcus aureus (“MRSA”) (Boyce, Potter-Bynoe, Chenevert, and King, 1997; Loftus et al. 2008), the inability of current infection control policies to eradicate hospital acquired infections (“HAI”), and the skyrocketing costs of HAI; have fueled debate on the adequacy of current recommendations, stimulated scientific investigation, and forced an emphasis on best practices (Williams, 2008). The Joint Commission on the Accreditation of Hospitals (“JCAHO”), the principal accrediting body for healthcare institutions, announced that infection prevention is among its highest priorities for 2009 (Soule, n.d.). Additionally, The Centers for Medicare and Medicaid Services (“CMS”), as a result of the Federal Government's Deficit Reduction Act of 2005, announced that they would no longer reimburse for many HAIs (CMS Fact Sheet, 2008), an action which has major implications for the economic viability of an already troubled healthcare system.

In The Cost of Nosocomial Infections (Press Ganey Associates, n.d.) report that hospital acquired infections add a mean cost of $13,973 to each hospital stay. Within the surgical arena, much has been achieved with the intent to maintain sterility of surgical instruments and the surgical field, and thereby protect the patient from potential pathogens and disease transmission. The 1996 National Nosocomial Infections Surveillance Report found that surgical site infections (“SSI”) still account for 17% of the total HAIs (as cited in Mauermann and Nemergut, 2006), and with SSI caused by MRSA the cost is $135,367. Engemann et al. (2003) describe the actual number of SSIs occurring each year in the United States as 500,000, and they estimate for those caused by MRSA the additional losses can approximate $92,363 (as cited by Nelson, Bivens, Shinn, Wanzer, and Kasper, 2009).

Hall (1994) reports that the ability of the inanimate surgical environment to transmit infectious diseases or pathogenic microorganisms; has not been the focus of much research. Kramer, Scwebke, and Kampf (2006, p. 2334) discuss “inanimate surfaces as a frequent source of outbreaks for nosocomial infections”. Mangum, Horan, Pearson, Silver, and Jarvis (1999, p. 247), report that, “environmental surfaces in the operating room are rarely implicated as sources of pathogens important in the development of SSIs”. Despite these disagreements, Williams (2008, p. 274) proposes that the goal of all perioperative practices should be to prevent environmental contamination and breaching the chain of infection. She notes that “without a clean environment, other infection control measures such as hand hygiene and decontamination procedures may be undermined”. Current recommendations are consistent in their mandate that contaminated surfaces be cleansed with an appropriate disinfectant, only whenever visible soiling with blood or body fluids is recognized (OSHA, 1991, section 1910.1030[d][4][ii]) (CDC, 1999). OSHA offers more stringent stipulations on disinfection in this same section with “Contaminated work surfaces shall be decontaminated with an appropriate disinfectant after completion of procedures; immediately or as soon as feasible when surfaces are overtly contaminated or after any spill of blood or other potentially infectious materials; and at the end of the work shift if the surface may have become contaminated since the last cleaning”.

However, Rowley and Dingwall (2007), citing multiple scientific studies, note the inability of current decontamination and sterilization procedures to remove all microbial and proteinaceous materials from surgical and anesthetic equipment. Multiple scientific studies have confirmed bacterial contamination and colonization of the surgical environment, pre and post disinfection (Hall, 1994; Nelson, Bivens, Sbinn, Wanzer, & Kasper, 2006). Madar, Novakova, & Baska (2005) found that inanimate objects tested for microbial growth, cultured bacteria (including MRSA) off of 92% of tested stethoscopes. The researchers also found pathogens on blood pressure cuffs, esmarch bandages, and refrigerator door handles. Boyce et al. (1997) discovered that out of 38 patients with MRSA infection or colonization, 27% of environmental surfaces in the patient rooms tested positive for MRSA.

When the results were controlled for body site of infection or colonization, 85% with a wound or urine infection had positive colonization of environmental surfaces in their rooms. Most notable of those surfaces were patient gowns, linens, overbed tables, and blood pressure cuffs. The concern of cross contamination is compounded by the ability of pathogens to survive for weeks or even months on inanimate surfaces (Kramer et al.).

Considerable evidence has also been acquired to implicate anesthesia equipment as reservoirs of bacterial colonization and blood contamination (Hall, 1994; Masylak et al, 2002; Simmons, 2000). Also noted as included in this area are the anesthesia machine, the supply cart(s), the surgical bed, and specialized equipment such as infusion pumps, warming devices, intravenous (IV) line poles, phones. See AANA Journal, June 2009, Vol. 77, No. 3, page 231.

However, to date, there are no national guidelines for recommendations regarding the decontamination of anesthesia equipment (King & Cooke, 2001). The American Society of Anesthesiologists (“ASA”) and the American Association of Nurse Anesthetists (“AANA”) address the issue in their respective publications (Recommendations for Infection Control for the Practice of Anesthesiology, 2^{nd} Edition) from the former and (The AANA Infection Control Guide, 2009) from the latter. Though similar, the recommendations from these professional anesthesia organizations differ in some respects. Confounding the lack of universal recommendations, Perry and Monaghan (2001) discuss problems with the design of anesthesia equipment that create difficulties with contaminant identification and disinfection. This research has led to the advent of disposable products for the anesthesia provider i.e. laryngoscope blades, oral airways, LMAs, and so forth. King and Cooke (2001) propose the idea of single-use anesthesia equipment to safeguard patients and staff against blood-borne diseases, while at the same time, installing a realistic method of achieving infection control mandates without hindering operating suite turnover time.

Despite mandates and recommendations for policies on disinfection of the operating room environment, the feasibility of this task is only surpassed by the non-compliance resulting from its impracticality (Boyce et al., 2007). Additionally, the redesign of equipment and the development of methods to protect equipment from blood contamination (which can serve as agars for microbial growth), achieve satisfactory levels of disinfection, and satisfy governmental and accrediting organization dictums is conspicuously absent.

No application currently exists to protect the accordion-type hoses of forced air warming units from such contamination, or to provide a practical method of disinfection for the hoses that do become soiled. Forced air warming units and the accordion-type hoses used therewith have become an integral part of anesthesia care. Arizant Medical, the manufacturer of the prototype forced air warming unit, claims that between introduction in 1987 through 1999, their device had been utilized in excess of 20 million times. With 26,000,000 anesthetics performed annually in the United States, and forced air warmers now used in the majority of these operations, the potential for contamination occurrences is staggering.

Because of the design of its accordion-type hose, the forced air warming unit is prone to problems with contaminant identification and proper disinfecting. The accordion-type hose is constructed with ridges and troughs, which allow the hose to bend and flex, and to some extent, stretch. The ridges and troughs form an irregular surface area along the length of the hose. The irregular surface area is extremely difficult, if not impossible to clean and/or disinfect properly. Buildup and contamination is especially prone to occur in the difficult to access troughs of the hose.

Moreover, surgical and anesthesia staff, under pressure to expedite operating room turnovers (Masylak et al., 2002), are unlikely to achieve a satisfactory level of disinfection. At a current cost of approximately $75, replacement after each case is not a cost effective or practical alternative. Therefore, the need for a practical and affordable solution is quite evident.

### BRIEF SUMMARY OF THE INVENTION

The inventive surgical and anesthesia conduit cover kit was devised to protect one of the most utilized and difficult to clean anesthetic devices; from contamination with blood or body fluids, and thereby eliminate it as a potential vector for the transmission of pathogens.

The inventive surgical and anesthesia conduit cover kit is a disposable barrier kit developed to protect the forced air warmer's hose from environmental contamination. In so doing, the inventive kit reduces costs incurred from unnecessary hose replacement, as well as man-hours lost on timely and imperfect disinfecting routines. More importantly, without the possibility of contamination by blood and body fluids, the equipment cannot serve as a potential reservoir for infectious and pathogenic microorganisms and potentially eliminates the danger to patients and to healthcare workers.

The inventive surgical and anesthesia conduit cover kit for infection control in the operating room environment comprises a tubing member sized to fit over a forced air warming unit accordion-type hose. The tubing member is sufficiently long to substantially cover the length of the accordion-type hose. A dispenser is provided to contain and dispense the tubing member. The dispenser includes fasteners for securing the tubing member to the accordion-type hose. The dispenser can be removably wall mounted or may be placed on a flat surface.

The tubing member can be provided in individual segments, sized according to the length of the accordion-type hose to be covered. The segments may be divided by cutting or with pre-fabricated perforations.

### BRIEF DESCRIPTION OF THE DRAWINGS

FIG. 1 is a perspective view of the inventive anesthesia conduit cover kit;

FIG. 2 is a perspective view showing the conduit cover as used on an accordion-type hose on a forced air warming unit with a patient;

FIG. 3 is a perspective view showing the conduit cover as used on an accordion-type hose;

FIG. 4 is a perspective view of a polymer snap type gripping fastener;

FIG. 4A is a perspective view of an open-ended polymer fastener;

FIG. 5 is a perspective view of a polymer ratcheting gripping fastener;

FIG. 6 is a perspective view of an alternative polymer snap type gripping fastener;

FIG. 7 is a perspective view of a hook and loop type strap fastener;

FIG. 8 is a perspective view of a zip tie;

FIG. 9 is a perspective view of a wall-mounted, flat frame dispenser;

FIG. 10 is a perspective view of a dispenser having a wire frame and separator;

FIG. 11 is a perspective view of a molded dispenser including a fastener container;

FIG. 12 is a perspective view of a low profile molded dispenser;

FIG. 13 is a perspective view of a dispenser having a serrated cutting edge and a fastener container; and

FIG. 14 is a perspective view of a dispenser having a slotted top and a fastener container.

### DETAILED DESCRIPTION OF THE INVENTION

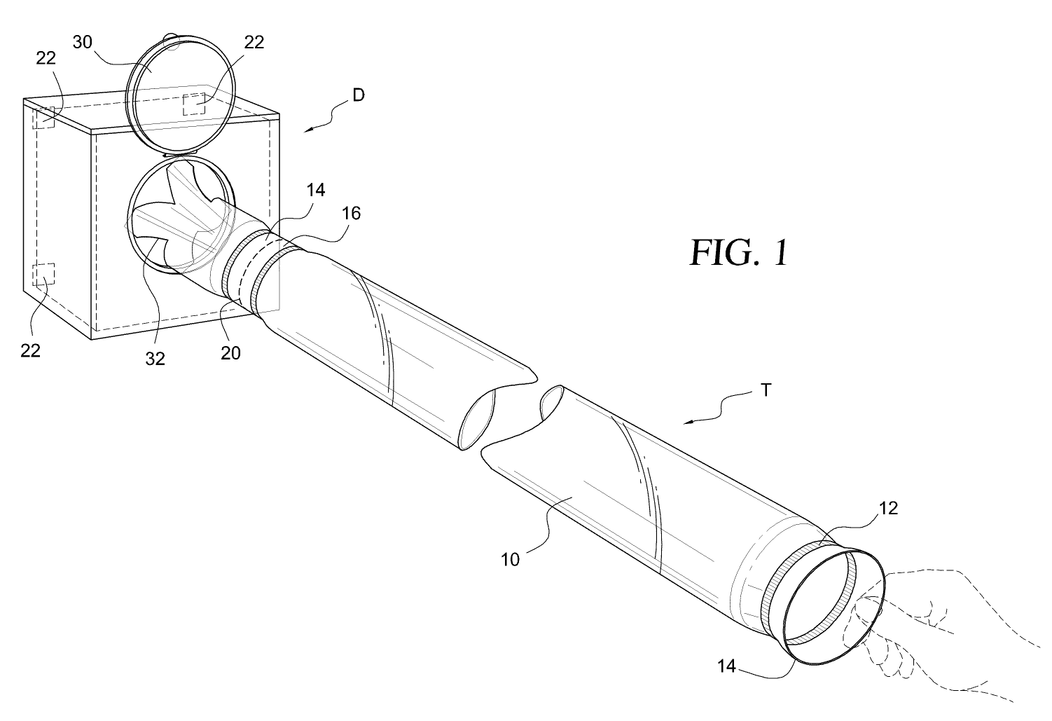

An embodiment of the inventive surgical and anesthesia conduit kit is shown in FIG. 1. A dispenser D contains a tubing member T, which is pulled from the dispenser opening 32. The tubing member T is typically separated into individual tubing segments 10. Each tubing segment 10 has a leading edge 14 and a trailing edge 16. A perforation 20 is positioned between the leading edge 14 and the trailing edge 16 that allows each tubing segment 10 to be individually separated. A cover 30 is provided which covers the dispenser opening 32 to maintain the tubing member T in a sanitary condition prior to extracting individual tubing segments 10 from the dispenser D.

The tubing member T is generally constructed of an anti-static polymer and is typically pink in color. In order to cover the diameter of the accordion-type hose 34, the tubing member T is typically approximately 5-7 inches in diameter and the wall of the tubing member T is typically approximately 2-6 mils thick.

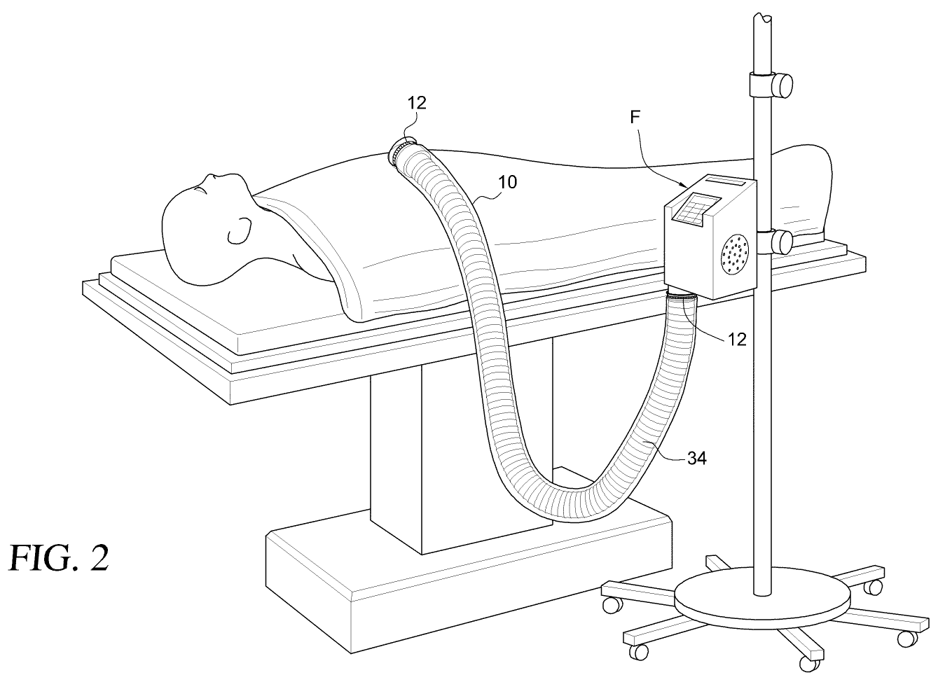

The length of the tubing segment 10 corresponds to the length of the accordion-type hose 34, which is used with a forced air warming unit F, as best seen in FIG. 2. The dispenser D can be left on a horizontal flat surface, such as a table or it may be wall mounted. In FIG. 1, hook and loop mounting pads 22 are shown. The hook and loop mounting pads 22 allow an empty dispenser D to be removed from the matching half of the hook and loop mounting pads 22 and a full, replacement dispenser D can be mounted on the wall on the same matching half of the hook and loop mounting pads 22.

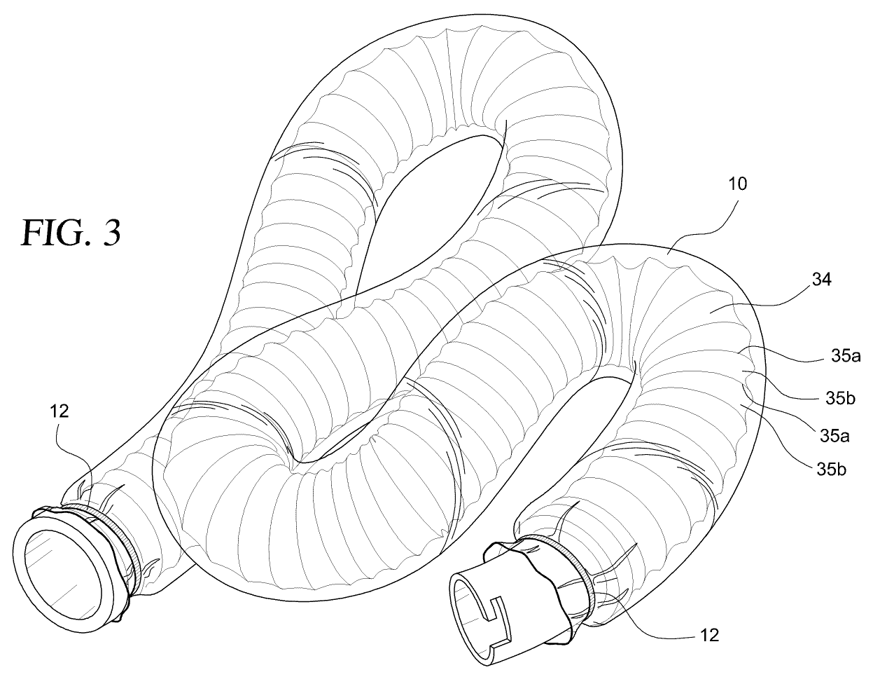

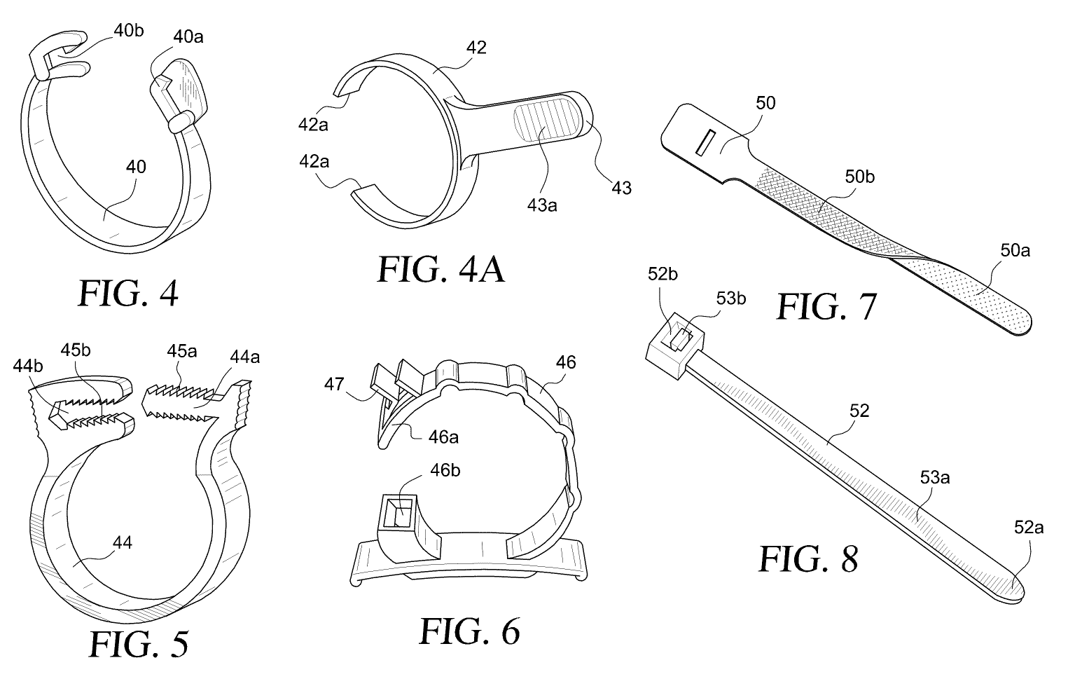

In FIG. 1 elastic fasteners 12 are shown on each end of the tubing segment 10. The elastic fasteners may be integral with our externally applied to the tubing segment 10. The elastic fasteners 12 secure the leading edge 14 and the trailing edge 16 to the accordion-type hose 34, as best seen in FIG. 3. Alternative fasteners may be used to secure the leading edge 14 and trailing edge 16 of the tubing segment 10. For example, in FIG. 4 a polymer snap type gripping fastener 40 is shown. The snap type gripping fastener 40 is non-adjustable and includes a tab 40a that engages a slot 40b to secure the fastener 40 to the tubing segment 10.

In FIG. 3, a detail of the accordion-type hose 34 is shown which shows the ridges 35a and troughs 35b. The ridges 35a and troughs 35b create irregular surfaces along the length of the accordion-type hose 34, which is very difficult or impossible to clean and/or disinfect properly.

In FIG. 4A an open-ended polymer fastener is shown that includes a resilient partial-circular band 42 having ends 42a. The ends 42a slip around the leading edge 14 and the trailing edge 16 of the tubing segment 10 to maintain a tight seal and to secure the tubing segment 10 to the accordion-type hose 34. When the open-ended fastener is removed, the ends 42a return to their original shape, which allows the open-ended fasteners to be reused. A handle 43 is attached to the partial-circular band 42, which allows easy application of the ends 42a onto the tubing segment 10 at the desired locations. Also included on the handle 43 is a gripping surface 43a to further simplify and enhance the ability of the user to grip the fastener handle 43. The partial-circular band 42 may also be made of flexible or resilient, non-polymer materials as desired.

In FIG. 5 a polymer ratcheting gripping fastener 44 is shown. Tab 44a includes serrated ridges 45a. Tab 44a engages slot 44b. Slot 44b includes serrated ridges 45b, which are engaged by the serrated ridges 45a on tab 44a. The ratcheting gripping fastener 44 can be adjusted to a limited extent by partially or fully engaging tab 44a with slot 44b.

In FIG. 6 an alternative polymer snap type gripping fastener 46 is shown. Tab 46a includes a resilient locking member 47. Tab 46a engages slot 46b and locking member 47 engages the interior of slot 46b to secure the fastener 46.

In FIG. 7 a hook and loop type strap fastener 50 is shown. Hooks 50a are on one side of the fastener 50 while loops 50b are on the other side of the fastener 50. When fastener 50 is wrapped around a tubing segment 10, the hooks 50a on one side of the fastener 50 engage the loops 50b on the other side of the fastener.

In FIG. 8 a zip tie fastener 52 is shown. The leading end 52a is inserted into slot 52b. Ridges 53a along the length of fastener 52 are retained in the slot 52b by a resilient clamping member 53b.

The anesthesia conduit kit may include any of the fasteners shown in FIGS. 1, 4-8 or an alternative desired fastener may also be used.

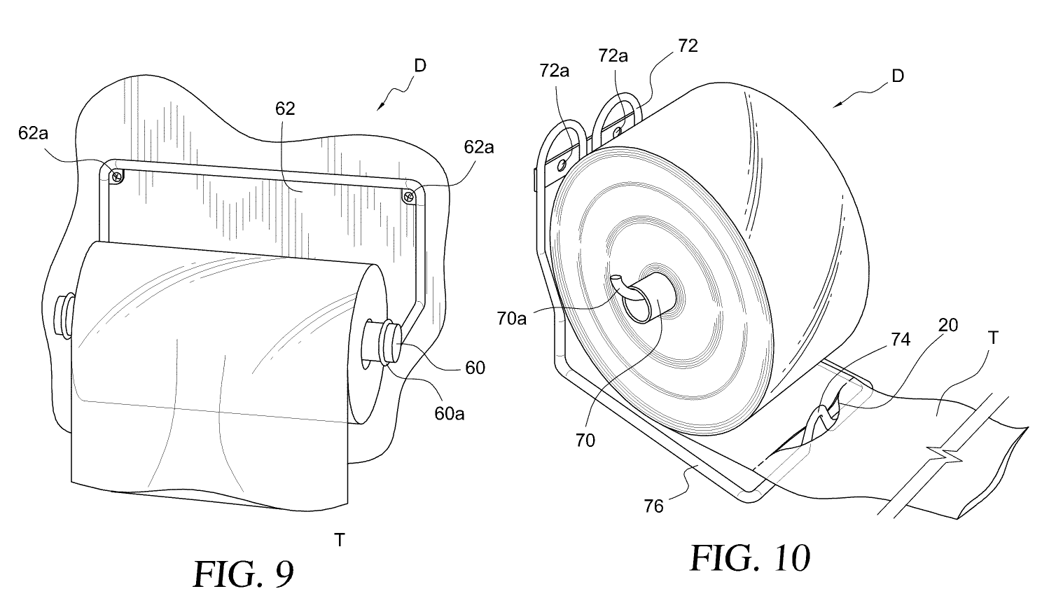

Although a box type dispenser D is shown in FIG. 1, alternative dispensers D are shown in FIGS. 9-14. FIG. 9 shows a wall-mounted dispenser that includes a rear frame 62 that is mounted to a wall surface with screws 62a or other fasteners. The tubing member T is on a rod 60, which is held by a loop 60a. Loop 60a is attached to the rear frame 62 to secure the tubing member T. Although not shown, the tubing member T may be divided into tubing segments 10, as shown in FIG. 1 with perforations 20, or the tubing member T may be continuous in length. A continuous length tubing member T can be cut to the desired length to fit on an accordion-type hose 34 or may also be used to cover any desired elongate member for which sanitary coverage is desired.

In FIG. 10 an alternative dispenser D is shown constructed of a wire frame 76. The wire frame 76 may be attached to a wall surface at the holes 72a or may be positioned on a flat surface, such as a table or other horizontal work surface. The tubing member T is on a tube 70, which is rotatable on rod 70a. The separator 74 is formed from a bend in the wire frame 76. The separator 74 allows the tubing member T to be separated easily at each perforation 20.

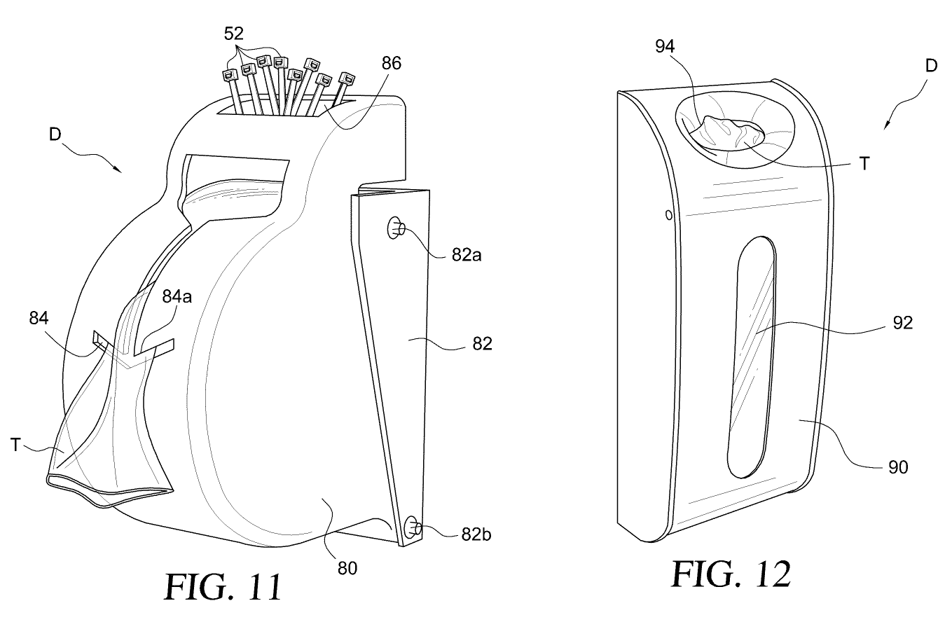

In FIG. 11 an alternative dispenser D is shown constructed of a molded body 80, which attaches to a wall mount frame 82 with pins 82a and 82b. A vertical slot 84a intersects a pair of upwardly angled slots 84b, 84c. A pointed edge 84d is formed at the intersection of the vertical slot 84a and the upwardly angled slot 84c. A pointed edge 84e is also formed at the intersection of vertical slot 84a and upwardly angled slot 84b. Each upwardly angled slot 84b, 84c is angled upwardly from a horizontal plane approximately 2-20 degrees. Each pointed edge 84d, 84e serve to separate the tubing member T into individual tubing segments 10. The tubing segments may be defined by perforations 20. Although two pointed edges 84d, 84e are provided, a single pointed edge, 84d or 84e could be used to separate the tubing member T into individual tubing segments 10. Two pointed edges 84d, 84e advantageously allow for separation of the tubing member T in either direction relative to the body 80. The tubing member T is on a spool 85, which is free to rotate within the body 80. Also molded into body 80 is a recessed container 86 to hold fasteners 52. Although fasteners 52 are shown, any desired fastener may be stored in container 86.

In FIG. 12 an alternative dispenser D is shown constructed of a molded body 90. Tubing member T is removed from opening 94 in the top of body 90. Opening 92 allows for an inspection of the contents of the body 90 to determine if it needs to be refilled. The tubing member T may include perforations 20 (not shown) or may be of continuous length. If tubing member T is of continuous length, it can be cut to fit the desired length to fit the accordion-type hose or any other elongate member. Fasteners 52 or other fastener types may be included in a separate container or they may be attached to the body 90.

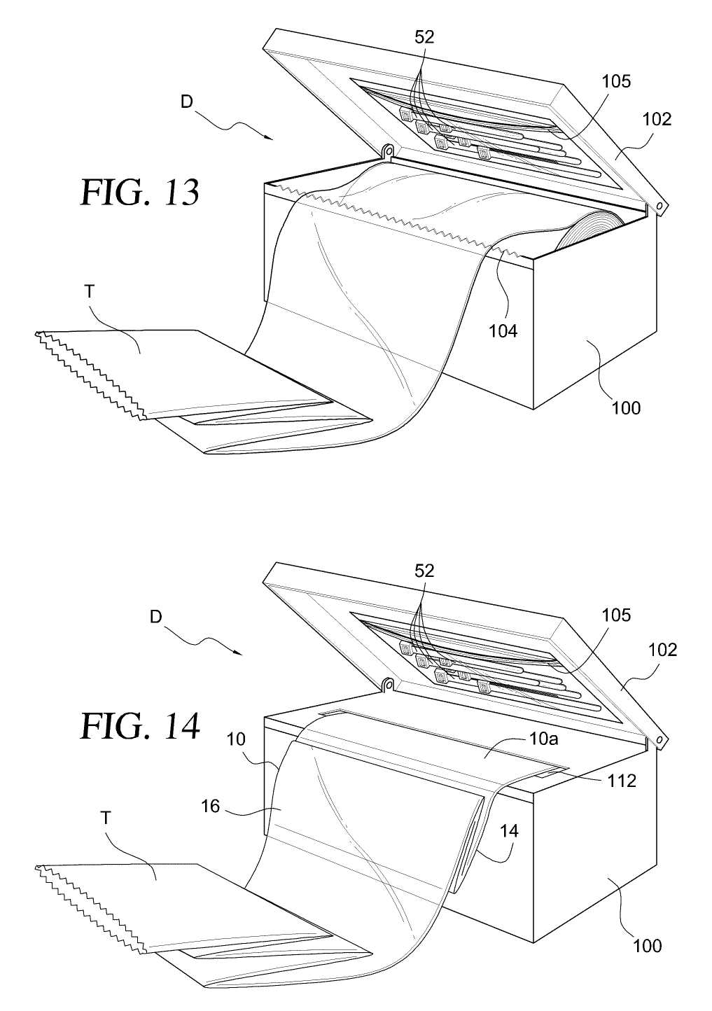

In FIG. 13 a dispenser D is shown comprising a box 100 having a sharpened edge 104 that is used to cut the tubing member T to the desired length. Cover 102 provided sanitation protection to keep the sharpened edge 104 and tubing member T covered. A container 105 on the inside of cover 102 provides ready access to fasteners 52 or any fasteners that are stored in the container 105.

FIG. 14 shows a dispenser having a box container 110, which includes a slot 112. The tubing member T is fed out of slot 112. Individual tubing segments 10 are shown overlapping between the trailing edge 16 of the leading segment 10 and the leading edge 14 of the following segment 10a, whereby the trailing edge 16 of each tubing segment 10 pulls the leading edge 14 of the next tubing segment 10a. A container 105 on the inside of cover 102 provides ready access to fasteners 52 or any fasteners that are stored in the container 105.

While several embodiments and elements of the invention have been shown and described, it should be understood that other variations and elements will be apparent to those skilled in the art. Also, although the invention is directed to use with accordion-type hoses on forced air warming units, it is contemplated that the invention may also be used on other elongate members such as cardiac monitoring leads and the like. Therefore, it will be understood that the embodiments and elements shown in the drawings and described herein are merely for illustrative purposes, and are not intended to limit the scope of the invention, which is defined by the claims, which follow.

What is claimed is:

1. A surgical and anesthesia conduit cover kit for infection control in the operating room environment, comprising:
a. a tubing member sized to fit over a forced air warming unit accordion-type hose;
b. said tubing member being sufficiently long to substantially cover the length of the accordion-type hose;
c. a dispenser to contain and dispense said tubing member; and
d. said dispenser includes fasteners for securing said tubing member to the accordion-type hose.

2. A kit as claimed in claim 1 wherein said tubing member is provided in a continuous length in said dispenser and wherein said dispenser includes a sharpened edge for cutting said tubing member into individual segments of a desired length, wherein said desired length corresponds substantially to the length of the accordion-type hose.

3. A kit as claimed in claim 1 wherein said fasteners comprise substantially resilient partial-circular bands having at least a pair of ends on an open end of said partial-circular band, wherein said ends slip around said tubing member to secure said tubing member to the accordion-type hose.

4. A kit as claimed in claim 1 wherein said tubing member is provided in segments of a predetermined length according to the length of the accordion-type hose to be covered.

5. A kit as claimed in claim 4 wherein each said segment is defined by perforations in said tubing.

6. A kit as claimed in claim 1 wherein said fasteners are selected from the group consisting essentially of: adjustable polymer clamps, non-adjustable polymer clamps, hook and loop straps and zip-ties.

7. A kit as claimed in claim 1 wherein said dispenser includes a reusable surface mounting system, whereby a full dispenser can replace an empty dispenser and be mounted on the same wall mount surface.

8. A kit as claimed in claim 7 wherein said reusable surface mounting system comprises hook and loop fasteners with adhesive backing with one side of said hook and loop fastener being attached to a back side of said dispenser and another side of said hook and loop fastener being attached to a wall mount surface.

9. A kit as claimed in claim 3 wherein each said tubing member includes a leading edge and a trailing edge and wherein each said trailing edge engages the leading edge of a subsequent tubing member to pull said subsequent tubing member toward an outlet of said dispenser.

10. A kit as claimed in claim 1 wherein said tubing is constructed of an anti-static polymer.

11. A kit as claimed in claim 1 wherein said tubing is pink in color.

12. A kit as claimed in claim 1 wherein said tubing is approximately 5-7 inches in diameter.

13. A kit as claimed in claim 10 wherein the wall of said tubing is approximately 2-6 mils thick.

14. A kit for infection control in the operating room environment, comprising:
a. a tubing member sized to fit over a forced air warming unit accordion-type hose;
b. said tubing member being separated into segments, each segment being of a length corresponding substantially to the length of the accordion-type hose;
c. a dispenser to contain and dispense said tubing member and said segments; and
d. each end of said segments includes a substantially integral resilient band for securing each said end to each end of the accordion-type hose.

15. A kit as claimed in claim 12 wherein each of said segments includes a leading and a trailing edge, and wherein each said trailing edge engages the leading edge of a subsequent segment to pull said subsequent segment toward an outlet of said dispenser.

16. A method of making a kit for infection control in the operating room environment, comprising the steps of:
a. providing a dispenser to contain and dispense a tubing member;
b. sizing said tubing member to fit over a forced air warming unit accordion-type hose;
c. sizing said tubing member to be sufficiently long to substantially cover the length of the accordion-type hose; and
d. providing fasteners for securing said tubing member to the accordion-type hose.

17. A method as claimed in claim 16 comprising the additional steps of
a. providing a sharpened edge on said dispenser; and
b. cutting said tubing into individual segments of a desired length according to the length of the accordion-type hose.

18. A surgical and anesthesia conduit cover kit as claimed in claim 1 wherein said dispenser includes a slot from which said tubing member is pulled; said slot having a substantially vertical portion which intersects at least one substantially upwardly angled portion; wherein at least one substantially pointed edge portion is formed at the intersection of said substantially vertical portion and said substantially upwardly angled portion of said slot for separating said tubing member into individual segments.

19. A surgical and anesthesia conduit cover kit as claimed in claim 18 wherein said substantially upwardly angled portion is angled upwardly in the range of approximately 5-20 degrees from a horizontal plane.

20. A surgical and anesthesia conduit cover kit as claimed in claim 18 wherein said dispenser includes a molded container for holding fasteners to be used to fasten each end of said segments.

21. A surgical and anesthesia conduit cover kit as claimed in claim 18 wherein said segments are defined by perforations in said tubing member.
